(19) United States
(12) Patent Application Publication
(10) Pub. No.: US 2014/0303254 A1
(43) Pub. Date: Oct. 9, 2014

(54) Process Of Manufacturing A Stable, Ready To Use Infusion Bag For An Oxidation Sensitive Formulation

(71) Applicant: InnoPharma Licensing LLC, Piscatway, NJ (US)

(72) Inventors: Tushar Hingorani, Piscataway, NJ (US); Kumaresh Soppimath, Monmouth Jn, NJ (US); Rekha Nayak, Belle Mead, NJ (US); Tushar Malkan, Somerset, NJ (US); Satish Pejaver, Bridgewater, NJ (US); Navneet Puri, Bridgewater, NJ (US)

(21) Appl. No.: 14/246,047

(22) Filed: Apr. 5, 2014

Related U.S. Application Data
(60) Provisional application No. 61/809,538, filed on Apr. 8, 2013.

(51) Int. Cl. B65B 55/14; A61K 31/167

(52) U.S. Cl. CPC B65B 55/14; A61K 31/167
USPC 514/629; 53/425

(57) ABSTRACT

A process for minimizing formation of a highest degradation product during moist heat sterilization of a drug solution of an oxidation susceptible active pharmaceutical ingredient is provided. The oxidation susceptible active pharmaceutical ingredient is mixed with excipients and deoxygenated water to prepare a non-sterile drug solution. The non-sterile drug solution is filled into a moist heat sterilizable flexible infusion bag. The flexible infusion bag with the non-sterile drug solution is terminally moist heat sterilized at a preset air overpressure between about 0.2 bar to about 1.2 bar to obtain a parenteral drug product. The highest degradation product in the parenteral drug product is less than 0.5% by weight of a labeled amount of the oxidation susceptible active pharmaceutical ingredient in the parenteral drug product.

### CROSS REFERENCE TO RELATED APPLICATIONS

This application claims priority to and the benefit of provisional patent application No. 61/809,538 titled “Process Of Manufacturing A Stable, Ready To Use Infusion Bag For An Oxidation Sensitive Formulation”, filed in the United States Patent and Trademark Office on Apr. 8, 2013. The specification of the above referenced patent application is incorporated herein by reference in its entirety.

### BACKGROUND

The drug product disclosed herein relates to a stable, ready to use parenteral drug product prepared by moist heat sterilization of a drug solution comprising an active pharmaceutical ingredient in a flexible infusion bag, wherein the active pharmaceutical ingredient in the drug product is susceptible to oxidation by ambient oxygen, light, or moisture.

Oxidation of inorganic and organic compounds occurs by a loss of electrons and a loss of a molecule of hydrogen. Alcohols, aldehydes, ketones, alkynes, alkenes, sulfides, thiols, carboxylic acids, benzoins, phenols, quinones, alkylbenzenes, imines, epoxides, catechols, ethers, and organometallics are examples of oxidizable functional groups. These functional groups are found in pharmaceutical compounds such as acetaminophen, acetylcysteine, amikacin sulfate, dopamine hydrochloride, promethazine hydrochloride, linezolid, and in classes of compounds such as amino acids.

Acetaminophen, also referred to as paracetamol or N-(4-hydroxyphenyl)acetamide, is a non-steroidal analgesic and an antipyretic widely used via various routes and is represented as shown in the formula below:

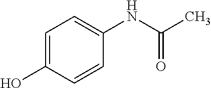

Acetaminophen administered by an intravenous route has a faster on-set and results in more predictable pharmacokinetics than oral or rectal acetaminophen formulations. In a study where six adult volunteers were given intravenous, oral, and rectal acetaminophen, the mean intravenous Cₘₐₓ observed was nearly two and four fold higher compared to administration by an oral route and a rectal route respectively. The intravenous treatment group showed consistently better on-set and higher peak plasma and cerebrospinal fluid (CSF) maximum concentration values with less variability than after either oral or rectal administration.

An advantage of intravenous acetaminophen is that the intravenous acetaminophen may be administered before or during surgery, permitting the initiation of an effective analgesic therapy in an early phase of a post-operative period. Intravenous acetaminophen appears to avoid first pass hepatic exposure and metabolism via portal circulation, which may reduce the potential for hepatic injury. With therapeutic dosing, for example, with up to 4,000 mg daily, intravenous acetaminophen is rarely associated with hepatotoxicity and has been shown to be safe for use in some patients with underlying liver conditions. Nonetheless, according to its prescribing information, intravenous acetaminophen is contraindicated in patients with severe hepatic impairment or severe active liver disease. Advantages of the acetaminophen injection are well known in the art.

Acetaminophen is a p-aminophenol derivative, which is synthesized by acetylation of p-aminophenol with acetic anhydride. Acetaminophen may be hydrolyzed to p-aminophenol at an elevated temperature and in the presence of an acidic medium or a basic medium. p-aminophenol is a major impurity in acetaminophen preparations that may be formed during the storage or synthesis of acetaminophen. It was reported that p-aminophenol may cause nephrotoxicity and teratogenicity; therefore, the amount of p-aminophenol should be strictly controlled. The United States and British pharmacopeias limit the amount of p-aminophenol in an acetaminophen substance at 0.005% w/w.

The degradation of acetaminophen in an aqueous solution is both an acid catalyzed reaction and a base catalyzed reaction. It is first order with respect to the concentration of acetaminophen and first order with respect to hydrogen and hydroxyl ion concentration. The half life for acetaminophen in a buffered solution at pH 5 and pH 6 was calculated to be 19.8 years and 21.8 years respectively. At pH 2, the half life is 0.73 years, and at pH 9, the half life is 2.28 years, with intermediate values at intermediate pH values. While formulating acetaminophen in pharmaceuticals, it is desirable to keep the pH of the medium between about 5 to about 6 to maximize the shelf life for the product.

In addition to hydrolysis, acetaminophen also undergoes oxidation decomposition that involves formation of a quinone-imine that may readily polymerize with generation of nitrogen-containing olegomers and polymers. These polymers, in particular, those stemming from N-acetyl-p-benzoquinone-imine are the toxic metabolite of acetaminophen, which is endowed notably with a cytotoxic and hemolytic effect. The decomposition of this metabolite in an aqueous medium is still more complex and gives rise to p-benzoquinone and hydroquinone. Some co-solvent compositions of acetaminophen contain ethanol and polyethylene glycol 400.

Acetaminophen is the active pharmaceutical ingredient in the marketed product Ofirmev® Injection of Cadence Pharmaceuticals, Inc. The Ofirmev® Injection is a sterile, clear, colorless, non-pyrogenic, isotonic formulation of acetaminophen intended for intravenous infusion. The Ofirmev® Injection has a pH of approximately 5.5 and an osmolality of approximately 290 milliosmole per kilogram (mOsm/kg). Each 100 mL contains 1000 mg acetaminophen, United States Pharmacopeia (USP), 3850 mg mannitol, USP, 25 mg cysteine hydrochloride monohydrate, USP, and 10.4 mg dibasic sodium phosphate, USP. The pH of the Ofirmev® Injection product is adjusted with hydrochloric acid and/or sodium hydroxide.

Terminal sterilization is the method of choice for sterilization of thermally stable active pharmaceutical ingredients. To achieve sterility of a non-sterile drug solution, the non-sterile drug solution must be sterilized in an autoclave to obtain a minimum 6 log reduction of microbial bioburden in the non-sterile drug solution. Each log reduction (10⁻¹) represents a 90% reduction in the microbial bioburden. Therefore, a process shown to achieve a “6 log reduction” (10⁻⁶) will reduce the microbial bioburden from a million organisms (10⁶) to very close to zero, theoretically. It is common to employ an overkill cycle to provide maximum assurance of sterility for critical products such as parenteral solutions, implantable devices, etc. The 6 log reduction is achieved by sterilizing the non-sterile drug solution for at least 15 minutes at 121° C. (250° F.) at 100 kPa (15 psig), or for at least 3 minutes at 134° C. (273° F.) at 100 kPa (15 psig). Additional sterilization time is typically required where the non-sterile drug solution and instruments are packed within an overwrap, as they may take longer to reach the required sterilization temperature. The acetaminophen active pharmaceutical ingredient is susceptible to oxidation. An autoclave cycle of acetaminophen in the presence of oxygen leads to the formation of dimer and polymeric impurities, where the acetaminophen drug solution is between a pH of about 5 to about 6. To minimize degradation of the active pharmaceutical ingredient and the generation of impurities during terminal sterilization, different approaches have been taken. In one approach, the water used for compounding the acetaminophen is deoxygenated and the acetaminophen drug solution is thereafter terminally sterilized in non-oxygen permeable glass bottles in the presence of antioxidants. Conventionally, fluids for parenteral administration to the blood stream of patients have been packaged in glass containers. However, manufacturing and transport of glass containers is challenging. Industrial efforts have been made to find alternative polymeric materials which are less resource consuming, cheaper, and more convenient to handle than glass. In one such effort, stabilization of the acetaminophen solution during terminal sterilization was carried out using an acetaminophen dimer.

Based on the package insert of the Ofirmev® Injection, it is recommended that each 100 mL glass vial contains 1000 mg acetaminophen (10 mg/mL). Once the vacuum seal of the glass vial has been penetrated, or when the contents are transferred to another container, it is recommended that the dose of the Ofirmev® Injection be administered within 6 hours.

There are several commercially available compositions of ready to use acetaminophen injection solutions containing propylene glycol as the only co-solvent, wherein the process for the preparation of a packaged, ready to use acetaminophen injectable solution comprises the following steps: mixing acetaminophen with water, propylene glycol as the only co-solvent, and a citrate buffer having a pH from 4.5 to 6.5; heating the resulting solution to a temperature between 70° C. and 130° C.; keeping the resulting solution at the same temperature for at least 10 minutes; aseptically packaging the acetaminophen in a container, and sterilizing the acetaminophen drug solution in the container to obtain a parenteral acetaminophen drug product.

Also, there are several commercially available ready to use intravenous infusion solutions of acetaminophen which contain acetaminophen, an aqueous solvent, a buffer to adjust the pH of the drug product to between 4.5 and 6.5, an isotonic agent, and a detectable amount of at least 0.005%, as a ratio of the surface area of peaks measured by high-performance liquid chromatography (HPLC) with detection at 245 nm, of an acetaminophen dimer.

Active pharmaceutical ingredients in a drug solution or in a drug product undergo degradation under various physical and chemical conditions, for example, during terminal sterilization of the non-sterile drug solution and yield impurities which adversely affect the performance of the active pharmaceutical ingredient. Hence, drug regulatory agencies require applicants of new and generic drugs to submit stability indicating data of active pharmaceutical ingredients when a new drug or generic drug application is submitted for approval. Hence, it is necessary to develop a stability indicating method for analysis of the drug product, the active pharmaceutical ingredient and its impurities. During manufacture of the parenteral acetaminophen drug product, the active pharmaceutical ingredient, that is, acetaminophen, contained in a drug solution in the flexible infusion bag undergoes degradation by the following: initial oxygen present in the flexible infusion bag when the non-sterile acetaminophen drug solution is filled in the flexible infusion bag; the oxygen that permeates through the flexible infusion bag during moist heat sterilization; and the 121° C. temperature at which the non-sterile acetaminophen drug solution is sterilized. United States Food And Drug Administration (FDA) regulations suggest the percentage degradation of the active pharmaceutical ingredient during such finished drug product manufacture comply with the United States Pharmacopeia (USP), or the International Conference on Harmonisation of Technical Requirements for Registration of Pharmaceuticals for Human Use (ICH) guidelines. In establishing degradation product acceptance criteria, a critical consideration is whether the level of the highest degradation product is specified in the USP. The level of the degradation product in the drug product is required to be within the level specified in the USP.

Generally, during processing of an oxidation susceptible active pharmaceutical ingredient to a parenteral dosage form, the active pharmaceutical ingredient undergoes degradation by heat to which the active pharmaceutical ingredient is exposed during terminal moist heat sterilization. ICH guidelines for parenteral formulations require the unknown impurity to be identified. The maximum allowable amount of the impurity depends on the concentration of the daily dose. For example, if the daily dose is greater than 2 gm per day, the identification limit is 0.1% by weight of the active pharmaceutical ingredient; if the daily dose is 1-10 mg, the identification limit is 0.5% by weight of the active pharmaceutical ingredient.

In a moist heat sterilization cycle, air overpressure is typically set at about 1.3 bar to about 1.4 bar to prevent the contents including the drug solution in the flexible infusion bag from expanding and bursting the flexible infusion bag during sterilization. Also, in a moist heat sterilization cycle of an oxidation susceptible active pharmaceutical ingredient at an air overpressure set at about 1.4 bar, the degradation of the oxidation susceptible active pharmaceutical ingredient may exceed 0.5% by weight of the labeled amount of the oxidation susceptible active pharmaceutical ingredient in the drug product in the parenteral dosage form.

Conventional formulations and processes use several excipients and packaging to stabilize the formulation, but fail to address degradation of the active pharmaceutical ingredient during terminal sterilization. Hence, there is a long felt but unresolved need for reducing the degradation of oxidation susceptible formulations during terminal moist heat sterilization. Furthermore, there is a need for a stable, oxidation susceptible drug solution contained in flexible infusion bags. Furthermore, there is a need for a process for manufacturing a stable, ready to use, oxidation susceptible drug product in a flexible infusion bag that precludes or reduces the oxidation and degradation of the oxidation susceptible active pharmaceutical ingredient during terminal moist heat sterilization.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The process disclosed herein addresses the above mentioned need for providing greater stability to a parenteral drug product that is made by moist heat sterilization of a non-sterile oxidation susceptible drug solution comprising an oxidation susceptible active pharmaceutical ingredient in a moist heat sterilizable container, for example, a flexible infusion bag.

An oxidation susceptible active pharmaceutical ingredient is mixed with excipients and a deoxygenated solvent, for example, deoxygenated water to prepare a non-sterile drug solution. The non-sterile drug solution is filled in a container, for example, a container made of a flexible material, referred herein as a “flexible infusion bag”. The fill volume of the flexible infusion bag is, for example, about 20 mL to about 1000 mL. The flexible infusion bag is moist heat sterilizable. The flexible infusion bag containing the non-sterile drug solution is thereafter terminally sterilized by moist heat sterilization in an autoclave at a preset air overpressure between about 0.2 bar to about 1.2 bar, for example, preset at 0.7 bar, to obtain a parenteral drug product, wherein the highest degradation product in the parenteral drug product is less than 0.5% by weight of the labeled amount of the oxidation susceptible active pharmaceutical ingredient. For example, the highest degradation product contains an impurity less than about 0.5% by weight of the oxidation susceptible active pharmaceutical ingredient. The parenteral drug product obtained after moist heat sterilization of the non-sterile drug solution in the flexible infusion bag is a stable, ready to use parenteral drug product. It was unexpectedly found that controlling the air overpressure in the autoclave at a pressure preset between about 0.2 bar and about 1.2 bar reduced the formation of the highest degradation product in the parenteral drug product to about 0.01% to 0.5% by weight of the labeled amount of the oxidation susceptible active pharmaceutical ingredient. In an embodiment, controlling the air overpressure in the autoclave at a pressure preset between about 0.2 bar and about 1.2 bar reduced the formation of the highest degradation product in the parenteral drug product to less than 0.5% by weight of the labeled amount of the oxidation susceptible active pharmaceutical ingredient. In an embodiment, the air overpressure in the autoclave during steam sterilization of the drug solution is maintained at less than about 1.2 bar.

In an embodiment, the flexible infusion bag filled with the non-sterile drug solution is enclosed within one or more overwraps to preclude the ingress of oxygen, moisture, and/or light to the non-sterile drug solution within the flexible infusion bag during terminal moist heat sterilization and post sterilization during storage of the drug product. The flexible infusion bag with or without an overwrap and with the non-sterile drug solution is moist heat sterilized to obtain a 6 log reduction, minimum, of microbial bioburden in the non-sterile drug solution in the flexible infusion bag.

In another embodiment, the flexible infusion bag filled with the non-sterile drug solution is moist heat terminally sterilized in an autoclave. The sterilized, flexible infusion bag with the oxidation susceptible drug solution is enclosed within an overwrap in a class 10,000 or class 100,000 clean room to preclude ingress of oxygen, moisture, and/or light into the oxidation susceptible drug product during storage of the drug product. In an embodiment, the sterilized, flexible infusion bag with the oxidation susceptible drug product is enclosed within an overwrap under a class 100 or a class 10,000 laminar flow hood in a class 100,000 clean room.

In an embodiment, the flexible infusion bag, or the flexible infusion bag with the overwrap containing the oxidation susceptible drug solution is sterilized by a water cascade sterilization method or steam sterilization at a temperature and a cycle time configured to obtain a minimum of 6 log reduction of the microbial bioburden in the oxidation susceptible drug solution, for example, sterilized at a minimum temperature of about 121° C. for a preset time of, for example, between about 10 minutes to about 30 minutes with an air overpressure set at a pressure between about 0.2 bar to about 1.2 bar, for example, about 0.7 bar.

In an embodiment, the oxidation susceptible active pharmaceutical ingredient is acetaminophen and the parenteral drug product disclosed herein is a parenteral acetaminophen drug product contained in a flexible infusion bag. The oxidation susceptible acetaminophen active pharmaceutical ingredient is mixed with excipients and deoxygenated water to prepare a non-sterile acetaminophen drug solution. The non-sterile acetaminophen drug solution is filled in a flexible infusion bag. The flexible infusion bag with the non-sterile acetaminophen drug solution is terminally sterilized by moist heat sterilization in an autoclave at a preset air overpressure between about 0.2 bar to about 1.2 bar, for example, preset at 0.7 bar, to obtain the parenteral acetaminophen drug product at the desired therapeutic concentration in the flexible infusion bag. It was unexpectedly found that controlling the preset air overpressure in the autoclave to between about 0.2 bar and about 1.2 bar, for example, controlling the preset air overpressure to about 0.7 bar, reduced the formation of the highest degradation product in the parenteral acetaminophen drug product to less than 0.5% by weight of the labeled amount of acetaminophen. The highest degradation product is a known impurity or an unknown impurity of the oxidation susceptible active pharmaceutical ingredient analyzed at an ultraviolet wavelength of about 244 nm of acetaminophen in the parenteral acetaminophen drug product.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific processes and structures disclosed herein. The description of a process step or a structure referenced by a numeral in a drawing carries over to the description of that process step or structure shown by that same numeral in any subsequent drawing herein.

FIG. 1 exemplarily illustrates a process for preparing a stable, ready to use parenteral drug product that reduces degradation of an oxidation susceptible active pharmaceutical ingredient in a non-sterile drug solution during moist heat sterilization.

FIG. 2 exemplarily illustrates a moist heat sterilizable flexible infusion bag provided with an overwrap containing an oxidation susceptible drug solution therewithin.

FIG. 3 exemplarily illustrates a chromatogram showing results of sterilizing a drug solution containing an oxidation susceptible active pharmaceutical ingredient, filled in a flexible infusion bag at an air overpressure, preset between 0.2 bar and 1.2 bar.

FIG. 4 exemplarily illustrates a table showing peak results of sterilizing a drug solution containing an oxidation susceptible active pharmaceutical ingredient, filled in a flexible infusion bag at an air overpressure, preset between 0.2 bar and 1.2 bar.

FIG. 5 exemplarily illustrates a chromatogram showing results of moist heat sterilizing an oxidation susceptible drug solution filled in a flexible infusion bag at preset air overpressures more than 1.2 bar.

FIG. 6 exemplarily illustrates a table showing peak results of moist heat sterilizing an oxidation susceptible drug solution filled in a flexible infusion bag at preset air overpressures more than 1.2 bar.

### DETAILED DESCRIPTION OF THE INVENTION

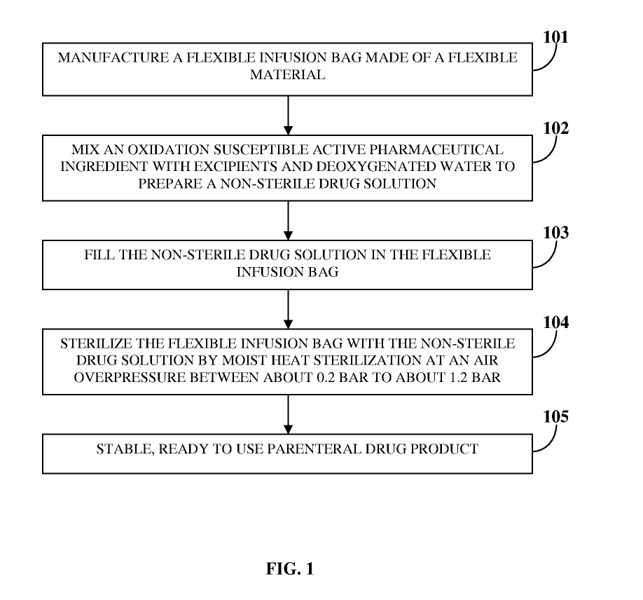

FIG. 1 exemplarily illustrates a process for preparing a stable, ready to use parenteral drug product that reduces degradation of an oxidation susceptible active pharmaceutical ingredient in a non-sterile drug solution during moist heat sterilization. As used herein, “active pharmaceutical ingredient” is any drug substance or mixture of drug substances used in the manufacture of a drug product and that, when used in the production of a drug, becomes an active ingredient in the drug product. Also, as used herein, “drug solution” refers to the non-sterile formulation of the active pharmaceutical ingredient and excipients prior to sterilization of the non-sterile formulation in a flexible infusion bag. Also, as used herein, “drug product” refers to the sterilized drug solution in a parenteral dosage form, for example, the sterilized drug solution in a flexible infusion bag. The drug solution is moist heat sterilized in a moist heat sterilizable container, herein referred as a “flexible infusion bag”. As used herein, “sterilization” refers to terminal sterilization of the non-sterile drug solution in a container to achieve a minimum 6 log reduction of microbial bioburden in the non-sterile drug solution.

In the process disclosed herein, a flexible infusion bag made of a flexible material, for example, a plastic material is manufactured 101. An oxidation susceptible active pharmaceutical ingredient is mixed 102 with one or more excipients and deoxygenated water to prepare a non-sterile drug solution. In an embodiment, the oxidation susceptible active pharmaceutical ingredient is acetaminophen. In an embodiment, the acetaminophen solution is an aqueous based isotonic solution comprising about 2 mM to about 500 mM of at least one of an acetate buffer, a citrate buffer, a borate buffer, a phosphate buffer, a maleic buffer, a succinic buffer, a tartaric buffer, a phthalate buffer, a formate buffer, a tris buffer, or any combination thereof. In an embodiment, the pH of the acetaminophen solution is between about 5 to about 6.

The non-sterile drug solution is then filled 103 in the flexible infusion bag. The fill volume of the flexible infusion bag is, for example, about 20 mL to about 1000 mL. The flexible infusion bag with the non-sterile drug solution in the flexible infusion bag is terminally sterilized 104 by moist heat sterilization in an autoclave at a preset air overpressure between about 0.2 bar to about 1.2 bar, for example, preset at 0.7 bar, to produce the stable, ready to use parenteral drug product 105. The air overpressure of the autoclave maintains degradation of the highest degradation product to less than 0.5% by weight, for example, between about 0.01% by weight to about 0.5% by weight of the labeled amount of the oxidation susceptible active pharmaceutical ingredient measured by a reverse phase high-performance liquid chromatography (HPLC) technique with a limit of quantitation of about 0.01%. The highest degradation product is a known impurity or an unknown impurity analyzed at an ultraviolet wavelength of, for example, about 244 nm.

In an embodiment, the flexible infusion bag filled with the non-sterile drug solution is enclosed within one or more overwraps. The flexible infusion bag with or without an overwrap and with the non-sterile drug solution is moist heat sterilized in an autoclave to achieve a 6 log reduction, minimum, of the microbial bioburden in the non-sterile drug solution. The overwrap reduces the degradation of the non-sterile drug solution in the flexible infusion bag by precluding or decreasing the ingress of oxygen, moisture, and/or light into the non-sterile drug solution during sterilization and after sterilization during storage of the drug product 105.

In another embodiment, the flexible infusion bag filled with the non-sterile drug solution is moist heat terminally sterilized in an autoclave. The sterilized, flexible infusion bag with the oxidation susceptible drug product is enclosed within an overwrap in a class 10,000 or class 100,000 clean room to preclude ingress of oxygen, moisture and/or light into the drug product in the sterilized flexible infusion bag during storage of the drug product 105. In an embodiment, the sterilized, flexible infusion bag with the oxidation susceptible drug product is enclosed within an overwrap under a class 100 or a class 10,000 laminar flow hood in a class 100,000 clean room.

The highest degradation product of the oxidation susceptible active pharmaceutical ingredient is less than 0.5% by weight of the labeled amount of the oxidation susceptible active pharmaceutical ingredient in the parenteral drug product 105 as measured by a reverse phase high-performance liquid chromatography (HPLC) method. In another embodiment, the highest degradation product is at a level of less than 0.05% by weight of the labeled amount of the oxidation susceptible active pharmaceutical ingredient in the parenteral drug product 105 as measured by the reverse phase HPLC method. In another embodiment, the highest degradation product is at a level of about 0.01% by weight of the labeled amount of the oxidation susceptible active pharmaceutical ingredient in the parenteral drug product 105.

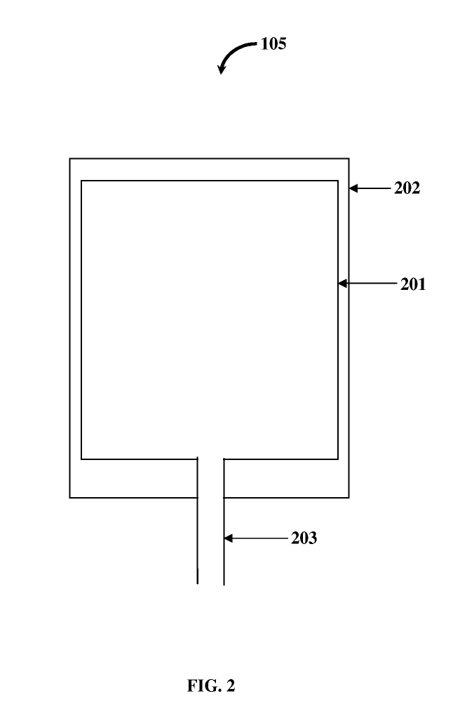

FIG. 2 exemplarily illustrates a moist heat sterilizable flexible infusion bag 201 provided with an overwrap 202 containing an oxidation susceptible drug solution (not shown) therewithin. The parenteral drug product 105 disclosed herein comprises a sterilized solution of an oxidation susceptible active pharmaceutical ingredient and one or more excipients contained within the flexible infusion bag 201. The flexible infusion bag 201 is filled with a non-sterile drug solution comprising the oxidation susceptible active pharmaceutical ingredient and sterilized in an autoclave, for example, by water cascade sterilization methods or steam sterilization methods under the following exemplary parameters: preset air overpressure between about 0.2 bar and about 1.2 bar, a sterilization temperature of about 121° C., and a sterilization cycle time between about 15 minutes to about 30 minutes to obtain a minimum of 6 log reduction of microbial bioburden in the drug solution. The stable, ready to use parenteral drug product 105 prepared is in a unit dose form having a volume of, for example, about 20 mL to about 1000 mL.

The stable, ready to use parenteral drug product 105 disclosed herein generally relates to a stable product of an oxidation susceptible drug solution comprising an oxidation susceptible active pharmaceutical ingredient in any suitable therapeutically effective amount, where the oxidation susceptible active pharmaceutical ingredient has one or more oxidizable functional groups comprising an alcohol, an aldehyde, a ketone, an alkyne, an alkene, a sulfide, a thiol, a carboxylic acid, benzoin, phenol, quinone, alkylbenzene, imines, epoxides, catechols, ethers, and organometallics. The stable, ready to use parenteral drug product 105 disclosed herein generally also relates to a stable product of the oxidation susceptible active pharmaceutical ingredient with one or more oxidizable functional groups in pharmaceutical compounds such as acetaminophen, acetylcysteine, amikacin sulfate, dopamine hydrochloride, promethazine hydrochloride, linezolid, oxytocin, etc. In an embodiment, the stable, ready to use parenteral drug product 105 disclosed herein comprises one oxidation susceptible active pharmaceutical ingredient along with one or more active pharmaceutical ingredients not susceptible to oxidation.

In an embodiment, the oxidation susceptible active pharmaceutical ingredient is acetaminophen and is present in a therapeutically effective amount for management alone or in combination with other active pharmaceutical ingredients to treat mild to moderate pain, management of moderate to severe pain with adjunctive opioid analgesics, and reduction of fever. Typically, the acetaminophen is present in an amount of, for example, about 0.01% to about 99% w/w of the total drug product.

The flexible infusion bag 201 is made of a cycloolefinic polymer, a polypropylene polymer, a polyvinyl chloride polymer, etc. An example of a flexible infusion bag 201 is the Technoflex® infusion bag of Technoflex Société Anonyme à Directoire. In an embodiment, the flexible infusion bag 201 comprises composite layers of one or more of a minimum of two polymeric materials. In another embodiment, the flexible infusion bag 201 comprises one or more than one compartment. In an embodiment, the flexible infusion bag 201 comprises one or more than one port 203.

The oxidation susceptible drug solution comprises one or more excipients. For example, a formulation of the oxidation susceptible drug solution comprises a vehicle. In an embodiment, the vehicle used is a mixture of a minimum of two solvents. In another embodiment, the vehicle comprises one or more of water, alcohols, glycols, dimethylacetamide N-methylpyrollidone, dimethyl sulfoxide, etc. In another embodiment, the excipients comprise, for example, one or more of water, alcohols, glycols, dimethylacetamide, N-methylpyrollidone, dimethyl sulfoxide, etc.

In an embodiment, the formulation of the oxidation susceptible drug solution comprises buffering excipients. In an embodiment, the buffering excipient comprises one or more of an acetate buffer, a citrate buffer, a borate buffer, a phosphate buffer, a maleic buffer, a succinic buffer, a tartaric buffer, a phthalate buffer, a formate buffer, and a tris buffer. In another embodiment, the buffers are present at a concentration of, for example, about 2 millimolar (mM) to about 500 mM. For example, the buffers are present at a concentration of about 80 mM, at about 40 mM, at about 20 mM, at about 10 mM, or at about 5 mM. For example, in an embodiment, the acetaminophen drug solution comprises about 2 mM to about 500 mM of at least one of an acetate buffer, a citrate buffer, a borate buffer, a phosphate buffer, a maleic buffer, a succinic buffer, a tartaric buffer, a phthalate buffer, a formate buffer, a tris buffer, or any combination thereof.

In an embodiment, the oxidation susceptible drug solution comprises tonicity excipients. In an embodiment, a formulation of the oxidation susceptible drug solution comprises, for example, one or more of about 0.1% to about 1.5% w/v of sodium chloride, about 0.1% to about 1.5% w/v of potassium chloride, about 0.1% to about 1.5% w/v of calcium chloride, about 1% to about 20% w/v of sugars such as dextrose, about 0.1% to about 10% w/v of propylene glycol, and about 0.1 to about 10% w/v of glycerol. The tonicity excipients are present in an amount to make the drug product isotonic to blood.

The pH of the oxidation susceptible drug solution is adjusted to a pH of between 1 and 14. For example, in an embodiment, the pH of the oxidation susceptible drug solution has a pH of between 4 and 8. In another embodiment, the pH of the oxidation susceptible drug solution has a pH between 5.40 and 5.60. In an embodiment, the pH of the acetaminophen drug solution is in a range of about 5 to about 6.

In an embodiment, the flexible infusion bag 201 is overwrapped within one or more overwraps 202 prior to sterilization of the oxidation susceptible drug solution in the flexible infusion bag 201. In an embodiment, the overwrap 202 is a barrier layer configured to reduce or preclude permeation of oxygen to the oxidation susceptible drug solution contained within the flexible infusion bag 201 during or after sterilization. In another embodiment, the overwrap 202 is a barrier layer configured to reduce or preclude permeation of moisture to the oxidation susceptible drug solution contained within the flexible infusion bag 201 during or after sterilization. In another embodiment, the overwrap 202 is a barrier layer, for example, a plastic foil or an aluminum foil configured to reduce or preclude permeation and ingress of light to the oxidation susceptible drug solution contained within the flexible infusion bag 201 during or after sterilization. In another embodiment, the overwrap 202 is a barrier layer, for example, an aluminum overwrap configured to preclude permeation and ingress of oxygen, moisture, and light. In an embodiment, the oxidation susceptible drug solution is filled in the flexible infusion bag 201 overwrapped with a minimum of one overwrap 202, for example, an aluminum overwrap along with a minimum of one oxygen scavenger such as D-100 FreshPax® of Multisorb Technologies, Inc., Pharmakeep® KH-500 of Mitsubishi Gas Chemical Company, Inc., etc. In another embodiment, the oxygen scavenger is in the form of a powder, canisters, sheets films, and packets. In another embodiment, the oxidation susceptible drug solution is filled in the flexible infusion bag 201 overwrapped with a minimum of one overwrap 202 along with a minimum of one moisture scavenger, for example, the Zoldine® moisture scavenger of the Dow Chemical Company, the Sylosiv® moisture scavenger of W. R. Grace & Co.-Conn., etc. In an embodiment, the moisture scavenger is in the form of a powder, canisters, sheets films, and packets. In an embodiment, the overwrap 202 is, for example, a Polialuvel® overwrap with an oxygen permeability of about <0.01 [cm³/(m²*d*bar)] and water vapor permeability of about <0.01 [g/(m²*d)], Wipf® AG of WIPF Management AG Corporation.

In an embodiment, the oxidation susceptible drug solution in the flexible infusion bag 201 is moist heat sterilized with a minimum of one overwrap 202. In another embodiment, the flexible infusion bag 201 containing the oxidation susceptible drug solution is moist heat sterilized with a minimum of one overwrap 202 or with one or more overwraps, wherein the overwrap 202 comprises one or more oxygen scavengers and/or moisture scavengers configured to provide a barrier to ingress of oxygen, moisture, and/or light to the oxidation susceptible drug solution within the flexible infusion bag 201.

In the stable, ready to use parenteral drug product 105 disclosed herein, the volume of the oxidation susceptible drug solution filled in the flexible infusion bag 201 is, for example, between about 10 mL and about 5000 mL. For example, the volume of the oxidation susceptible drug solution is between about 50 mL and about 1000 mL. In another embodiment, the volume of the oxidation susceptible drug solution in the flexible infusion bag 201 is between about 80 mL and about 120 mL. In an embodiment, the strength of the acetaminophen in the parenteral acetaminophen drug product is 10 mg/mL.

In an embodiment, the flexible infusion bag 201 filled with the oxidation susceptible drug solution is terminally sterilized by moist heat sterilization at a minimum temperature of about 80° C. In another embodiment, the flexible infusion bag 201 filled with the oxidation susceptible drug solution is terminally sterilized by moist heat at a minimum temperature of about 90° C. In another embodiment, the flexible infusion bag 201 filled with the oxidation susceptible drug solution is terminally sterilized by moist heat at a minimum temperature of about 100° C. In another embodiment, the flexible infusion bag 201 filled with the oxidation susceptible drug solution is terminally sterilized by moist heat at a minimum temperature of about 121° C. for a time period between about 5 minutes and about 20 minutes.

In an embodiment, the oxidation susceptible drug product disclosed herein is a ready to use, parenteral solution of acetaminophen, wherein the highest degradation product in the acetaminophen drug product is less than 0.5% by weight of the labeled amount of acetaminophen in the parenteral acetaminophen drug product. In an embodiment, the highest degradation product in the ready to use parenteral acetaminophen drug product is not more than about 0.1% of any highest impurity, for example, not more than about 0.08% of any highest impurity, not more than about 0.050% of any highest impurity, not more than about 0.035% of any highest impurity, or not more than about 0.010% of any highest impurity of the oxidation susceptible acetaminophen active pharmaceutical ingredient in the parenteral acetaminophen drug product.

### Example 1

The constituents of formulations I to X of an acetaminophen drug solution are shown in the table below. The choice of a buffering agent has an impact on the formation of impurity during the autoclave cycle.

| | Concentration (mg/mL) | | | | | | | | | |
|---|---|---|---|---|---|---|---|---|---|---|
| Component | I | II | III | IV | V | VI | VII | VIII | IX | X |
| Acetaminophen | 10 | 10 | 10 | 10 | 10 | 10 | 10 | 10 | 10 | 10 |
| Sodium | — | 6.4 | 5.2 | 0.58 | — | — | — | — | — | — |
| Chloride | | | | | | | | | | |
| Citric Acid | — | 1.92 | 3.54 | 15.37 | — | — | — | — | — | — |
| Acetic Acid | — | — | — | — | — | — | — | 4.8 | 1.15 | 0.57 |
| Sodium Acetate | — | — | — | — | — | — | — | — | — | — |
| Disodium | — | — | — | — | 0.48 | 2.68 | 1.34 | — | — | — |
| Phosphate | | | | | | | | | | |
| Monosodium | — | — | — | — | 10.58 | 0.08 | 0.04 | — | — | — |
| Phosphate | | | | | | | | | | |
| Monohydrate | | | | | | | | | | |
| Sodium | q.s | 0.86 | 1.79 | 8 | — | — | — | 2.82 | 0.69 | 0.34 |
| Hydroxide | | | | | | | | | | |
| pH | 5.5 | 5.5 | 5.5 | 5.5 | 5.5 | 5.5 | 5.5 | 5.5 | 5.5 | 5.5 |

Based on the table above, consider examples of the following formulations:

Formulation I comprises 10 mg/mL of acetaminophen with a pH of 5.5.
Formulation II comprises 10 mg/mL of acetaminophen, 6.4 mg/mL of sodium chloride, 1.92 mg/mL of citric acid, and 0.86 mg/mL of sodium hydroxide, with a pH of 5.5.
Formulation III comprises 10 mg/mL of acetaminophen, 5.2 mg/mL of sodium chloride, 3.54 mg/mL of citric acid, and 1.79 mg/mL of sodium hydroxide, with a pH of 5.5.
Formulation IV comprises 10 mg/mL of acetaminophen, 0.58 mg/mL of sodium chloride, 15.37 mg/mL of citric acid, and 8 mg/mL of sodium hydroxide, with a pH of 5.5.
Formulation V comprises 10 mg/mL of acetaminophen, 0.48 mg/mL of disodium phosphate, and 10.58 mg/mL of monosodium phosphate monohydrate, with a pH of 5.5.
Formulation VI comprises 10 mg/mL of acetaminophen, 2.68 mg/mL of disodium phosphate, and 0.08 mg/mL of monosodium phosphate monohydrate, with a pH of 5.5.
Formulation VII comprises 10 mg/mL of acetaminophen, 1.34 mg/mL of disodium phosphate, and 0.04 mg/mL of monosodium phosphate monohydrate, with a pH of 5.5.
Formulation VIII comprises 10 mg/mL of acetaminophen, 4.8 mg/mL of acetic acid, and 2.82 mg/mL of sodium hydroxide, with a pH of 5.5.
Formulation IX comprises 10 mg/mL of acetaminophen, 1.15 mg/mL of acetic acid, and 0.69 mg/mL of sodium hydroxide, with a pH of 5.5.
Formulation X comprises 10 mg/mL of acetaminophen, 0.57 mg/mL of acetic acid, and 0.34 mg/mL of sodium hydroxide, with a pH of 5.5.

Formulations I-X tabulated above were manufactured on a lab scale. Water was deoxygenated by bubbling nitrogen until the dissolved oxygen content in the water was found to be less than 1 part per million (ppm). About 50 percent of the final water required for the drug product 105 was taken in a compounding vessel. Salt and buffering excipients were added to the compounding vessel and mixed with deoxygenated water until completely dissolved. The pH of the solution was adjusted to 5.5 with sodium hydroxide and hydrogen chloride (HCl). Separately, a weighed quantity of acetaminophen was mixed with about 20 percent of the final quantity of deoxygenated water for about 1 hour until a slurry was formed. This slurry was added to the main compounding vessel and the temperature of the bulk was increased to about 35° C. to 45° C. by heating. Mixing was continued until a clear solution was obtained. The heat was turned off and the non-sterile drug solution was allowed to cool to room temperature. The final volume was made up and filled in 20 mL glass vials. The non-sterile drug solution was moist heat sterilized in a Tuttnauer® Brinkmann® autoclave at 121° C. for 30 minutes and placed on stability at 60° C.

Stability Results:

| | Time Point (Days) | | | | | | | |
|---|---|---|---|---|---|---|---|---|
| Formu- | Acetaminophen Assay | | | | Highest Unknown Impurity (%) | | | |
| lation | 0 | 1 | 2 | 9 | 0 | 1 | 2 | 9 |
| I | 100.0 | 99.92 | 99.90 | 99.55 | 0 | 0.17 | 0.28 | 1.32 |
| II | 100.0 | 99.96 | 99.97 | 99.79 | 0 | 0.08 | 0.12 | 0.67 |
| III | 100.0 | 99.99 | 99.97 | 99.62 | 0 | 0.06 | 0.10 | 0.51 |
| IV | 100.0 | 99.99 | 99.97 | 99.72 | 0 | 0.07 | 0.10 | 0.44 |
| V | 100.0 | 99.94 | 99.89 | 99.65 | 0 | 0.16 | 0.30 | 1.01 |
| VI | 100.0 | 99.94 | 99.90 | 99.49 | 0 | 0.15 | 0.30 | 1.51 |
| VII | 100.0 | 99.84 | 99.95 | 99.73 | 0 | 0.11 | 0.17 | 0.79 |
| VIII | 100.0 | 99.82 | 99.60 | 98.53 | 0 | 0.42 | 1.11 | 4.07 |
| IX | 100.0 | 99.93 | 99.77 | 98.93 | 0 | 0.27 | 0.69 | 3.04 |
| X | 100.0 | 99.89 | 99.80 | 99.04 | 0 | 0.25 | 0.59 | 2.78 |

Conclusion: The formulations containing the phosphate buffer had a maximum amount of impurity. Unbuffered and acetate buffered solutions had similar amounts of impurities. Citrate buffered solutions had a minimum amount of impurities post autoclaving, and at the end of nine days, the citrate buffered solutions had the lowest impurities among the formulations studied.

The highest unknown impurity at two different preset air overpressures during a steam sterilization cycle is given below:

| Air overpressure | Highest Unknown Impurity (wt %) |
|---|---|
| 0.3 psig | 0.022 |
| 0.6 psig | 0.063 |

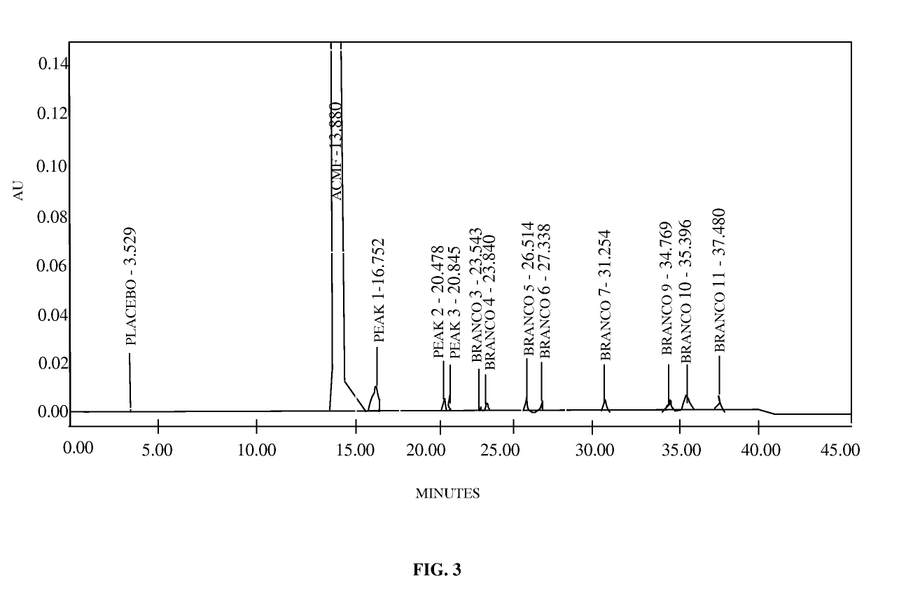

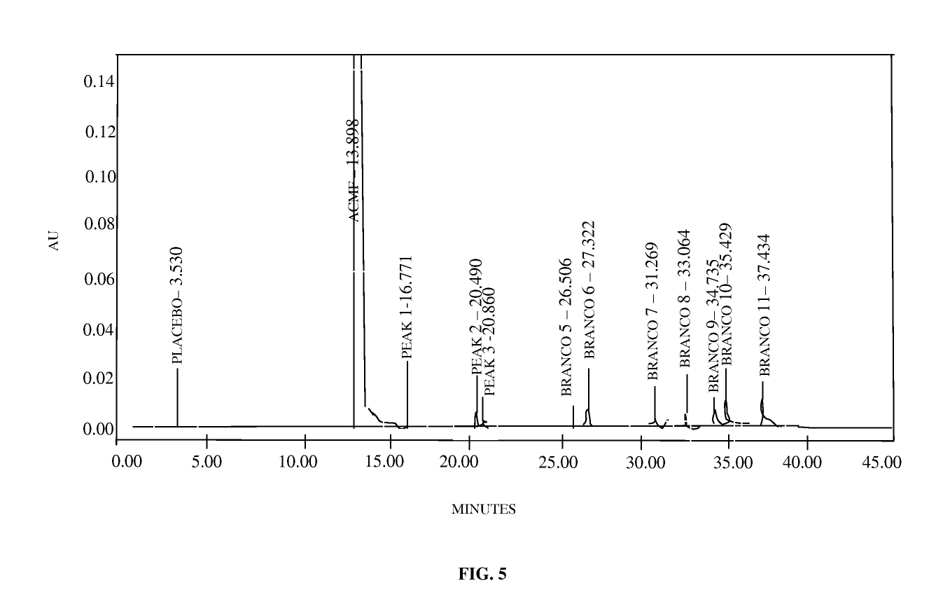

Formulation II was manufactured as described above. Formulation II was filled in polypropylene infusion bags with a polypropylene twist off port. The polypropylene infusion bags were then autoclaved in a Fedegari FOB3 steam sterilizer using different air overpressures. Lower preset air overpressures produce less impurity and lower impurity levels compared to the same formulation when autoclaved in a bottle at a higher preset air overpressure of about 1.2 bar. FIG. 3 and FIG. 5 exemplarily illustrate chromatograms with low preset air overpressure and high preset air overpressure respectively.

FIG. 3 exemplarily illustrates a chromatogram showing results of sterilizing a drug solution containing an oxidation susceptible active pharmaceutical ingredient, filled in a flexible infusion bag 201 exemplarily illustrated in FIG. 2, at an air overpressure, preset between 0.2 bar and 1.2 bar. A test chromatogram of related substances obtained from a sample sterilized at about 0.2 bar is exemplarily illustrated in FIG. 3.

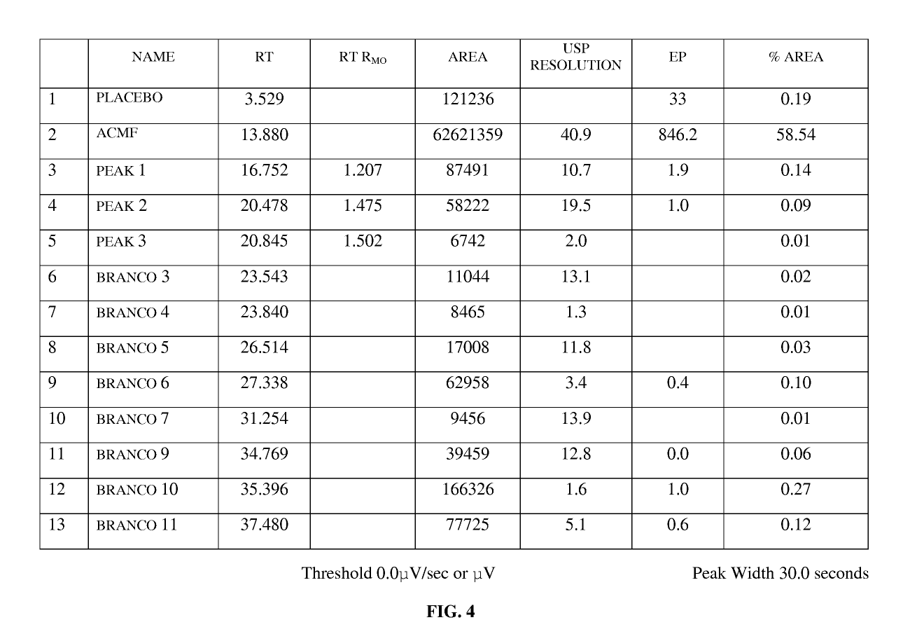

FIG. 4 exemplarily illustrates a table showing peak results of sterilizing a drug solution containing an oxidation susceptible active pharmaceutical ingredient, filled in a flexible infusion bag 201 exemplarily illustrated in FIG. 2, at an air overpressure, preset between 0.2 bar and 1.2 bar. FIG. 4 shows the highest unknown impurity at a retention time of about 16.8 minutes (RRT about 1.21) is about 0.03% when measured against an external dilute standard of acetaminophen.

FIG. 5 exemplarily illustrates a chromatogram showing results of moist heat sterilizing an oxidation susceptible drug solution filled in a flexible infusion bag 201 exemplarily illustrated in FIG. 2, at preset air overpressures more than 1.2 bar. FIG. 5 shows a test chromatogram of related substances obtained from a sample sterilized at about 1.2 bar.

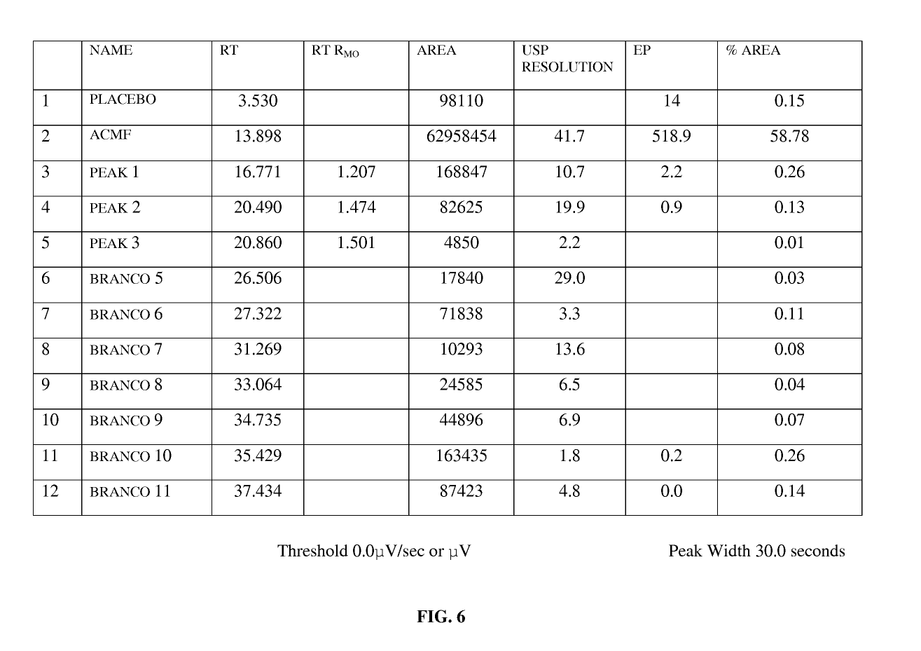

FIG. 6 exemplarily illustrates a table showing peak results of moist heat sterilizing an oxidation susceptible drug solution filled in a flexible infusion bag 201 exemplarily illustrated in FIG. 2, at preset air overpressures more than 1.2 bar. As shown in FIG. 6, the highest unknown impurity at a retention time of about 16.8 minutes (RRT about 1.21) is about 0.06% when measured against an external dilute standard of acetaminophen.

### Example 2

Formulations II, III and IV were prepared as disclosed above and filled in polypropylene infusion bags with a polypropylene twist off port. The polypropylene infusion bags were then autoclaved in a steam sterilizer with a preset air overpressure of 0.3 bar. The polypropylene infusion bags were then packaged, for example, in a Polialuvel® aluminum overwrap with an oxygen scavenger such as D-100 FreshPax® of Multisorb Technologies, Inc., and placed on stability.

| For- | | | | | | Highest |
|---|---|---|---|---|---|---|
| mula- | Temper- | | | Osmo- | | Unknown |
| tion | ature | Time Point | pH | lality | Assay | (%) |
| IV | 40° C. | Pre-Autoclaved | 5.61 | 292 | 101.0 | ND |
| | | Initial | 5.52 | 294 | 100.7 | 0.016 |
| | | 1 Wk | 5.54 | 291 | 101.2 | 0.015 |
| | | 2 Wk | 5.52 | 292 | 100.3 | 0.016 |
| | | 1 Month | 5.54 | 288 | 102.9 | 0.016 |
| III | | Pre-Autoclaved | 5.55 | 299 | 99.7 | ND |
| | | Initial | 5.50 | 296 | 99.0 | 0.032 |
| | | 1 Week | 5.51 | 295 | 99.9 | 0.031 |
| | | 2 Week | 5.50 | 295 | 99.1 | 0.038 |
| | | 1 Month | 5.52 | 293 | 101.9 | 0.036 |
| II | | Pre-Autoclaved | 5.52 | 293 | 98.6 | ND |
| | | Initial | 5.49 | 293 | 98.6 | 0.022 |
| | | 1 Week | 5.51 | 289 | 99.4 | 0.023 |
| | | 2 Week | 5.50 | 292 | 99.1 | 0.029 |
| | | 1 Month | 5.50 | 289 | 101.9 | 0.045 |
| IV | 25° C. | Initial | 5.52 | 294 | 100.7 | 0.016 |
| | | 1 Month | 5.55 | 289 | 103.6 | 0.016 |
| III | | Initial | 5.50 | 296 | 99.7 | 0.032 |
| | | 1 Month | 5.50 | 293 | 101.2 | 0.032 |
| II | | Initial | 5.49 | 293 | 98.6 | 0.022 |
| | | 1 Month | 5.50 | 289 | 101.8 | 0.022 |

Conclusion: Formulations II, III and IV as disclosed in Example 2 above had low levels of impurity post autoclaving compared to vials, and the levels of impurity did not increase on stability. Packaging in an aluminum overwrap with an oxygen scavenger decreases impurity level during the stability studies.

### Example 3

### Formulation XI

| Component | |
|---|---|
| Acetaminophen | 5000 g |
| Citric Acid | 960 g |
| Sodium Chloride | 3105 g |
| Sodium Hydroxide | pH 5.5 |
| Water for Injection q.s. | 500 L |

Consider another example with the formulation XI above. About 90% of the total water required, for example, about 450 L was charged in a compounding vessel. Water was deoxygenated by bubbling nitrogen until an oxygen content of ≦0.5 ppm was achieved. A weighed quantity of citric acid was added and mixed until the weighed quantity of citric acid dissolved completely. Sodium chloride was then added to the compounding vessel and mixed until the sodium chloride completely dissolved. The pH of the solution was adjusted to 5.5 using 5N NaOH or 0.1N HCl. The solution was heated to a temperature between 35° C. to 45° C. Acetaminophen was added to the compounding vessel and mixed until the acetaminophen completely dissolved and the heat was turned off. The volume was made up using previously deoxygenated water to produce the non-sterile drug solution. The non-sterile drug solution (100 mL) was filled in a polypropylene infusion bag and sealed using a polypropylene twist off port. A water cascade autoclave Aquatherm® 3310 was used to run the following autoclave cycles to terminally sterilize the polypropylene infusion bag.

| Autoclave | | | | |
|---|---|---|---|---|
| Air Over- | | Sterilization Time | | |
| pressure | | 15 minutes | 20 minutes | 25 minutes |
| 0.5 bar | pH | 5.49 | 5.49 | |
| | Highest | 0.026 | 0.030 | |
| | Unknown | | | |
| | Total Impurities | 0.045 | 0.051 | |
| 0.8 bar | pH | 5.49 | 5.50 | |
| | Highest | 0.034 | 0.042 | |
| | Unknown | | | |
| | Total Impurities | 0.056 | 0.069 | |
| 1.3 bar | pH | 5.50 | 5.51 | 5.54 |
| | Highest | 0.044 | 0.058 | 0.078 |
| | Unknown | | | |
| | Total Impurities | 0.073 | 0.091 | 0.121 |

The formation of the unknown impurity was found to be dependent on time and on the preset air overpressure used. Preset air overpressure of about 0.1 bar to about 1.3 bar is used in the autoclave cycle during the sterilization phase and cooling phase to prevent bursting of the polypropylene infusion bag. The preset air overpressure should not have any bearing on the formation of an unknown impurity during the autoclave cycle since sterilization is mainly dependent on time and temperature used for sterilization. From the table above, it can be seen that at 15 minute and 20 minute sterilization times, the unknown impurity increases with the increase in preset air overpressure used for the sterilization cycle.

The use of the terms “a” and “an” and “the” and similar referents in the context of describing the invention, especially in the context of the following claims, are to be construed to cover both the singular and the plural, unless otherwise indicated herein or clearly contradicted by context. The terms “comprising”, “having”, “including”, and “containing” are to be construed as open ended terms, that is, meaning “including, but not limited to”, unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All processes described herein can be performed in any suitable order unless otherwise indicated herein or otherwise clearly contradicted by context. The use of any and all examples, or exemplary language, for example, “such as” provided herein, is intended merely to better illuminate the invention and does not pose a limitation on the scope of the invention unless otherwise claimed. No language in the specification should be construed as indicating any non-claimed element as essential to the practice of the invention.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials, and embodiments, the invention is not intended to be limited to the particulars disclosed herein; rather, the invention extends to all functionally equivalent structures, processes and uses, such as are within the scope of the appended claims. Those skilled in the art, having the benefit of the teachings of this specification, may affect numerous modifications thereto and changes may be made without departing from the scope and spirit of the invention in its aspects. Moreover, any combination of the above described elements in all possible variations thereof is encompassed by the invention unless otherwise indicated herein or otherwise clearly contradicted by context.

We claim:

1. A process for minimizing formation of a highest degradation product during moist heat sterilization of a drug solution comprising an oxidation susceptible active pharmaceutical ingredient in a moist heat sterilizable container, said process comprising:
sterilizing said drug solution in said moist heat sterilizable container by said moist heat sterilization at an air overpressure between about 0.2 bar to about 1.2 bar to obtain a parenteral drug product with a degradation of less than 0.5% by weight of a labeled amount of said oxidation susceptible active pharmaceutical ingredient.

2. The process of claim 1, wherein said moist heat sterilizable container is a flexible infusion bag made of a plastic material, wherein said plastic material is one of a cycloolefinic polymer, a polypropylene polymer, a polyvinyl chloride polymer, and any combination thereof.

3. The process of claim 1, wherein fill volume of said moist heat sterilizable container is about 20 mL to about 1000 mL.

4. The process of claim 1, wherein said drug solution with oxidizable functional groups comprises an alcohol, an aldehyde, a ketone, an alkyne, an alkene, a sulfide, a thiol, a carboxylic acid, benzoin, phenol, quinone, alkylbenzene, imines, epoxides, and organometallics.

5. The process of claim 1, wherein said oxidation susceptible active pharmaceutical ingredient is selected from the group consisting of acetaminophen, acetylcysteine, amikacin sulfate, dopamine hydrochloride, promethazine hydrochloride, linezolid, and oxytocin.

6. The process of claim 1, wherein said drug solution is an acetaminophen solution.

7. The process of claim 6, wherein said acetaminophen solution is an aqueous based isotonic solution.

8. The process of claim 6, wherein said acetaminophen solution contains about 2 mM to about 500 mM of a buffering agent.

9. The process of claim 8, wherein pH of said acetaminophen solution is about 5 to about 6.

10. The process of claim 6, wherein said acetaminophen solution comprises about 2 mM to about 500 mM of at least one of an acetate buffer, a citrate buffer, a borate buffer, a phosphate buffer, a maleic buffer, a succinic buffer, a tartaric buffer, a phthalate buffer, a formate buffer, a tris buffer, and any combination thereof.

11. The process of claim 1, wherein said moist heat sterilizable container is enclosed within one or more overwraps.

12. The process of claim 11, wherein said one or more overwraps comprise one or more of oxygen scavengers and moisture scavengers.

13. The process of claim 11, wherein said one or more overwraps are configured to provide a barrier to ingress of one or more of oxygen, moisture, and light to said drug solution within said moist heat sterilizable container.

14. The process of claim 1, wherein said moist heat sterilization of said moist heat sterilizable container with said drug solution is performed by one of water cascade sterilization and steam sterilization.

15. The process of claim 1, wherein said moist heat sterilization of said moist heat sterilizable container with or without an overwrap and with said drug solution is performed at a temperature and a time configured to obtain a minimum of 6 log reduction of microbial bioburden in said drug solution.

16. The process of claim 1, wherein said moist heat sterilization of said moist heat sterilizable container with or without an overwrap and with said drug solution is performed at a minimum temperature of about 121° C.

17. The process of claim 1, wherein said moist heat sterilization of said moist heat sterilizable container with or without an overwrap and with said drug solution is performed for a time of about 10 minutes to about 30 minutes.

18. The process of claim 1, wherein said highest degradation product contains an impurity of about 0.01% to about 0.5% by weight of said oxidation susceptible active pharmaceutical ingredient.

19. A parenteral drug product comprising:
a solution of an oxidation susceptible active pharmaceutical ingredient and one or more excipients, which is sterilized by a moist heat sterilizer at an air overpressure between about 0.2 bar to about 1.2 bar; and
a highest degradation product less than 0.5% by weight of a labeled amount of said oxidation susceptible active pharmaceutical ingredient.

20. A process for preparing a stable, ready to use parenteral drug product by minimizing formation of a highest degradation product during moist heat sterilization of a drug solution comprising an oxidation susceptible active pharmaceutical ingredient, said process comprising:
manufacturing a moist heat sterilizable container;
filling an oxidation susceptible drug solution in said manufactured moist heat sterilizable container; and
sterilizing said oxidation susceptible drug solution filled in said manufactured moist heat sterilizable container in an autoclave at an air overpressure between about 0.2 bar to about 1.2 bar to obtain said stable, ready to use parenteral drug product, wherein a highest degradation product in said stable, ready to use parenteral drug product is less than 0.5% by weight of a labeled amount of said oxidation susceptible active pharmaceutical ingredient.

21. The process of claim 20, wherein said moist heat sterilizable container is a flexible infusion bag.

22. The process of claim 20, wherein said moist heat sterilizable container is made of a plastic material, wherein said plastic material is a one of a cycloolefinic polymer, a polypropylene polymer, a polyvinyl chloride polymer, and any combination thereof.

23. The process of claim 20, wherein said oxidation susceptible drug solution comprises an active pharmaceutical ingredient with oxidizable functional groups comprising an alcohol, an aldehyde, a ketone, an alkyne, an alkene, a sulfide, a thiol, a carboxylic acid, benzoin, phenol, quinone, alkylbenzene, imines, epoxides, and organometallics.

24. The process of claim 20, wherein said oxidation susceptible active pharmaceutical ingredient is selected from the group consisting of acetaminophen, acetylcysteine, amikacin sulfate, dopamine hydrochloride, promethazine hydrochloride, linezolid, and oxytocin.

25. The process of claim 20, wherein said oxidation susceptible drug solution is an acetaminophen solution comprising acetaminophen as an active pharmaceutical ingredient.

26. The process of claim 25, wherein said acetaminophen solution comprises about 2 mM to about 500 mM of at least one of an acetate buffer, a citrate buffer, a borate buffer, a phosphate buffer, a maleic buffer, a succinic buffer, a tartaric buffer, a phthalate buffer, a formate buffer, a tris buffer, and any combination thereof.

27. The process of claim 26, wherein pH of said acetaminophen solution is about 5 to about 6.

28. The process of claim 20, wherein said moist heat sterilizable container comprises a plastic material overwrapped with one or more overwraps, wherein said one or more overwraps are configured to provide a barrier to ingress of one or more of oxygen, moisture, and light to said oxidation susceptible drug solution within said moist heat sterilizable container.

29. The process of claim 20, wherein said moist heat sterilizable container is enclosed within one or more overwraps comprising one or more oxygen scavengers.

30. The process of claim 20, wherein said moist heat sterilizable container filled with said oxidation susceptible drug solution is sterilized in a moist heat sterilizer by one of water cascade sterilization and steam sterilization.

31. The process of claim 20, wherein said moist heat sterilizable container filled with said oxidation susceptible drug solution is moist heat sterilized at a temperature and a time configured to obtain a minimum of 6 log reduction of microbial bioburden in said oxidation susceptible drug solution.
